(19) United States
(12) Patent Application Publication
(10) Pub. No.: US 2012/0220662 A1
(43) Pub. Date: Aug. 30, 2012

(54) PHYTOCEUTICAL COMPOSITIONS FOR PETS

(75) Inventors: Alfredo Gonzalez Perez, Mexico City (MX); Ali Jesus Asaff Torres, Hermosillo (MX); Maria Lucia Alejo Castillo, Zapopan (MX)

(73) Assignee: BIOKAB, S.A. DE C.V., Mexico City (MX)

(21) Appl. No.: 13/391,381

(22) PCT Filed: Dec. 15, 2009
(86) PCT No.: PCT/IB2009/007764

§ 371 Date: May 7, 2012

(30) Foreign Application Priority Data
Aug. 20, 2009 (MX) MX/A/2009/08889

(51) Int. Cl. A61K 31/192; A61P 35/00

(52) U.S. Cl. 514/570

(57) ABSTRACT

This invention comprises applying phytoceutical compositions to pets, which given their sedentary life and nutrition, are susceptible to a higher prevalence of cancer and degenerative diseases. The compounds contained in said compositions frequently have anti-oxidizing properties acting to inhibit harmful reactions of free radicals; free radicals are often a cause of cancer. Said compositions comprise extracts or concentrates obtained from the enzymatic or alkaline hydrolysis of several plant sources, containing effective concentrations of two or more phenolic compounds, such as ferulic acid, coumaric acid, caffeic acid, protocathecoic acid, and vanillic acid.

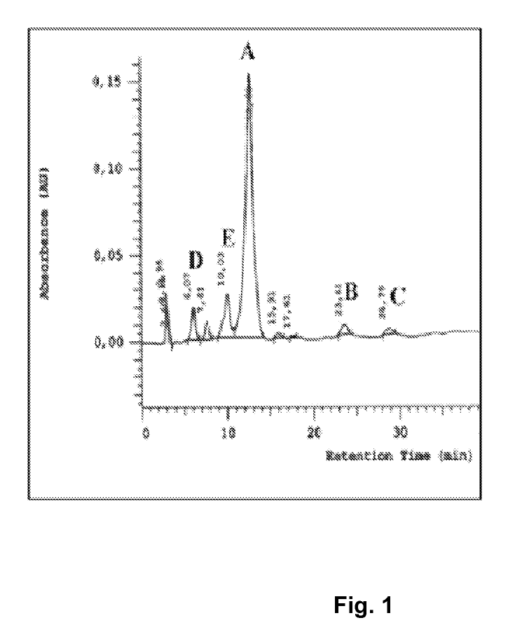

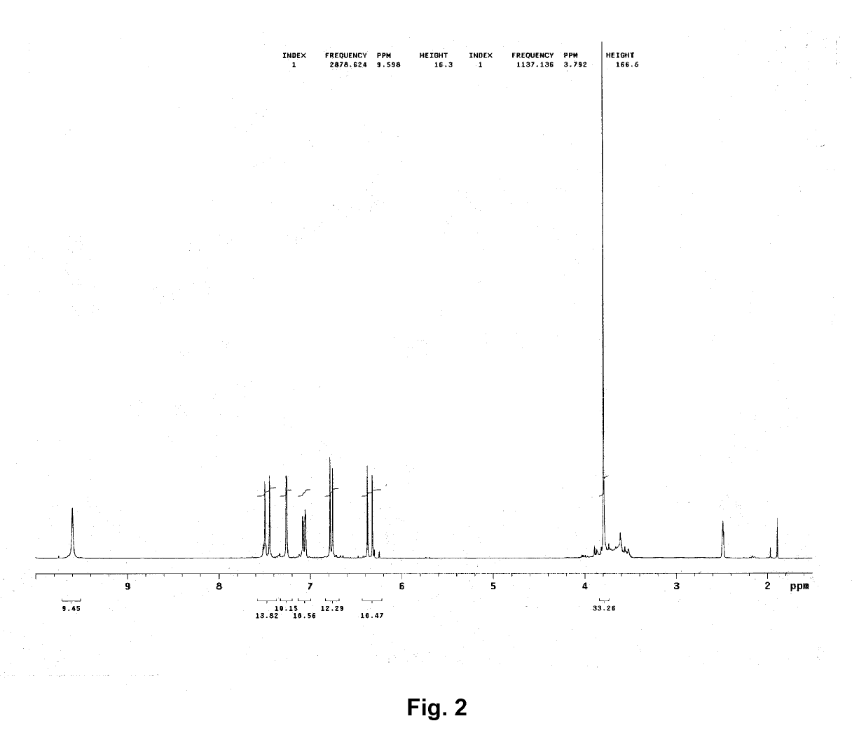

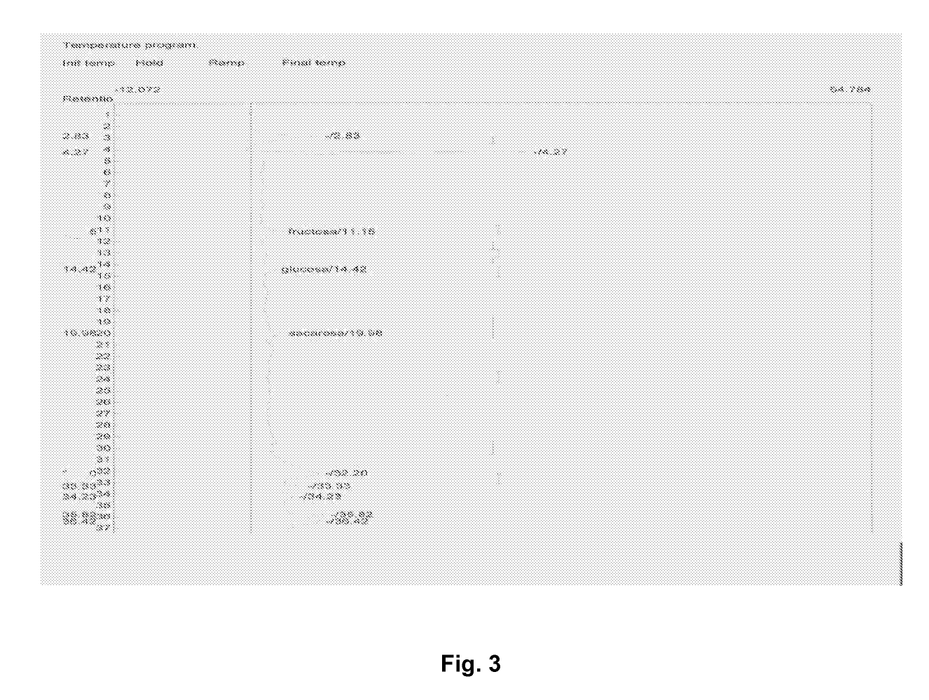

### CROSS-REFERENCE TO RELATED APPLICATIONS

This application is the U.S. National Stage under 35 U.S.C. §371 of International Patent Application No. PCT/IB2009/007764 filed Dec. 15, 2009, which claims the benefit of Mexican Patent Application No. MX/a/2009/008889 filed Aug. 20, 2009, entitled “Phytoceutical Compositions For Pets.”

STATEMENT REGARDING FEDERALLY SPONSORED RESEARCH OR DEVELOPMENT

Not applicable.

### FIELD OF THE INVENTION

The invention is associated with phytoceutical formulations used for the prevention and/or enhancement of their physiologic functions and their physical condition. The formulations comprise alkaline or enzymatic extracts from different plants, such as certain compounds which are found in the cooking liquor called nejayote in Spanish, and obtained from corn grains, strengthened with concentrates of ferulic acid crystals obtained from said cooking liquor (nejayote) containing said ferulic acid and other relevant active ingredients, having a synergic or additive activity in pets in order to prevent cancer tumors.

### PRIOR ART

Like humans, pets often suffer from different conditions. It is reported that, in the case of dogs, their susceptibility to suffer from cancer is 4 times higher than humans, although only 50% of tumors are malignant. It is estimated that one out of every 10 pets will have developed a kind of tumor throughout their lives, especially from 8 years of age. It is also common, especially in molosser breeds of dogs, to develop testicle cancer in males and breast and cervical cancer in females, as well as joint conditions such as arthritis characterized by the degeneration of joint cartilage and the formation of osteophytes. Another cancer frequently developed by shorthaired dogs is skin cancer, such as the one noted in Boxers from 7 years of age.

Apart from the prevalence of certain cancers in some particular breeds of dogs, the problem is more serious for most breeds due to the sedentary conditions in which they live and the mass type of nutrition for dogs, based on croquettes, a plurality of which contain elevated amounts of carcinogenic or teratogenic agents given their origin.

Keeping an optimal body weight is of the essence in order to prevent joint conditions. Intake of food containing anti-inflammatory compounds may be highly beneficial in the treatment and/or prevention of the disease. Cardiovascular diseases, oral diseases, colds, diseases resulting from liver damage caused by several factors, are also some of the common conditions in pets. These conditions have been exacerbated in recent years due to the wide diversity of harvests used in the industry of balanced foods, which harvests have a marked influence in the development of pathologies in pets.

A plurality of compounds which are capable of decreasing the risk of suffering these diseases or which are used for the treatment thereof are naturally occurring. These include flavonoids, stilbens such as resveratrol, lignans and phenolic acids, derivates of cinnamic acid, including but not limited to coumaric acid, protocathecoic acid, caffeic acid, sinapinic acid, vanillic acid, and ferulic acid. Most of these compounds have anti-oxidizing properties due to their ability to provide hydrogens from phenolic groups to free radicals. The ability of the ferulic acid to decrease reactive types of oxygen has also been reported, performing thereby the same role as superoxide dismutases, which are the enzymes that protect living creatures against reactive types of oxygen.

It is well known that all the degenerative reactions take place within the organism through free radical reactions. Due to this, if this oxidation mechanism is controlled through the donation of protons, the reaction will stop and many subsequent negative effects will not occur. Having this proton-donating capacity, ferulic acid is a good oxidizer and hence, it can prevent free radical reactions. Due to these properties, ferulic acid is mentioned in the list of food additives as an “oxidation inhibitor”, which may be used as an anti-oxidizing agent or anti-whitener agent in a plurality of patents. Several in vitro and in vivo studies on humans, animals and cell cultures have provided evidence of its multiple physiologic properties, such as: 1) lung, colon, liver, stomach and tongue cancer inhibition or prevention; 2) bone strengthening; 3) decrease of high lipid, triglyceride and glucose levels in blood, thus preventing thrombosis; 4) cholesterol production reduction; 5) preventing cell membrane damage due to free radicals; 6) immune system stimulation; 7) retina cell growth stimulation in degenerative retina diseases, as described by Fazzary and Ju; Acta Biochimica et Biophysica Sinica 2007, 39: 811-828. Ferulic acid has also been mentioned as an agent which: 8) protects the liver against toxic compounds; 9) protects the muscular system against wear; 10) prevents colds and flu given its antibiotic properties; 11) has anti-inflammatory properties and 12) has ergogenic properties by promoting muscular development (WO 2008/ 116319 A1).

Ferulic acid and other derivates of cinnamic acid widely occur in nature by forming a part of the cell wall in many vegetable species, such as corn, rice, wheat, barley, oat, sugar cane, sugar beet, coffee, tomato, vegetables, citric fruits, etc.

Three methods may be used to extract ferulic acid from its natural sources: 1) low molecular weight ferulic acid compounds; 2) cell wall of plant cells; 3) tissue cultures or microbial fermentations. Ferulic acid may also be chemically synthesized from vanilla condensation and malonic acid, producing thereby a mixture of cis and trans isomers. In the first two cases, hydrolysis methods are used, whether alkaline or enzymatic. From the hydrolysis products obtained, recovery and purification processes are then performed.

To date, the properties of cinnamic acid derivates have been studied from pure compounds. However, the mixtures of active ingredients contained in alkaline or enzymatic plant extracts, such as the ones contained in nejayote, and concentrates, allow us to achieve effective doses associated with the size and weight of the animal, which had not been studied before. This invention comprises an application or use of these mixtures acting in a synergic fashion.

### SUMMARY OF THE INVENTION

One of the purposes of the invention is to provide a natural phytoceutical composition containing a concentration of at least one cinnamic acid derivate, such as trans-ferulic acid, to prevent and/or treat some pet diseases.

A second goal of the invention is to provide a natural phytoceutical composition containing an effective concentration of at least one derivate of cinnamic acid, such as trans-ferulic acid, to improve physical condition in pets, promoting muscular development, bone strengthening and preventing wear in these systems.

A third goal of the invention is to provide a natural phytoceutical composition which contains an effective concentration of at least two derivates from cinnamic acid, searching for a synergic or additive activity to prevent and/or treat diseases and/or to improve physical condition in pets.

A fourth goal is to provide a method which benefits pet health by using natural products, which natural products, even if abundant, are not usually found occurring in nature and are to be obtained by hydrolysis, so they can then be concentrated and/or formulated with appropriate excipients.

### DESCRIPTION

Plant materials such as corn, rice, cane bagasse, oat, wheat, barley, sugar beet, tomato, citric fruits, vegetables, grass, stubble or other materials containing ferulic acid and/or other cinnamic acid derivates, are subject to an alkaline or enzymatic hydrolysis process, usually applying a cocktail comprising feruloyl esterases, xylanases and cellulases of fungal or bacterial sources. Concretely for corn, an ancestral practice in Mexico is nixtamalization, which consists in cooking the grains in a lime solution. This process allows for a partial hydrolysis of carbohydrates, releasing as well other nutrimental substances such as niacin and other substances considered to be nutraceutic, such as protocathecoic acid, coumaric acid, vanillic acid, ferulic, dimers and trimers thereof, in concentrations raging from 0.5 mg/L to 1.5 g/L, such as trans-ferulic acid.

A usual composition of nejayote in cinnamic acid derivates is the following: ferulic acid 1.2 g/L, coumaric acid 0.15 g/L, protocathecoic acid 0.03 g/L, as main active ingredients determined for HPLC according to FIG. No. 1., wherein: A) ferulic acid; E) coumaric acid; D) protocathecoic acid; B) and C) ferulic acid dimers.

The nature of some compounds was also revealed through a proton Nuclear Magnetic Resonance analysis (NMR) and a carbon-13 NMR, according to FIG. No. 2.

Besides, nejayote contains up to 35 g/L of solids in a solution conformed mainly by: fructose, sucrose, dextrins, glucose, starches, organic compounds (dissolved solids) proteins, soluble and insoluble fiber and some organic substances such as Ca, SO₄, Mg, etc. Having a bromatologic analysis with the properties shown in FIG. No. 3.

To provide an effective phytoceutical composition, the active ingredients contained in any type of hydrolysis product may be concentrated using a plurality of single operations such as:
1) any type of evaporation;
2) reverse osmosis;
3) adsorption/desorption;
4) any type of drying;
5) combinations of all of the above.

Liquid or solid concentrates resulting from the previous processes may be mixed to reach an optimal ratio of the active ingredients contained therein or marketed separately. In some cases, strongly pigmented compounds of dark colors, usually formed in the alkaline hydrolysis products, may be removed from the extracts through a selective adsorption with resins pertaining to the group of the amberlite, thus making the fenolic compounds available for use as main active ingredients.

Fluid compositions comprising this invention contain from 4 to 15 g/L of ferulic acid, whereas solid compositions comprise from 2 to 10% (wt./wt.) of this compound, considering the active ingredients as main benchmark.

The compositions of this invention do not attempt to purify the active ingredients contained in the extracts, since obtaining a synergic or additive activity of these compounds is intended in order to enhance the health of pets.

### EXAMPLES

### Example 1

An emulsion obtained through thermal-alkaline hydrolysis of white corn and rice husk in a ratio of 4:1 (w/w). This composition contains: 3 g/L ferulic acid, 0.2 g/L coumaric acid, 0.12 g/L vanillic acid, protocathecoic acid <50 mg/L, as active ingredients; furthermore, said composition contains other nutritional elements such as dextrins and starches 20 g/L, Vitamin A 0.35 μg/100 g, Vitamin C <14.00 mg/100 g, proteins 4 g/L. Said composition was administered locally into a group of 3 Boxer breed dogs, all from a litter of 9 individuals with an age of 7 years, which individuals suffered from skin cancer conditions and the result being that said cancers did not spread. Furthermore, the overall life quality of the pets improved in a remarkable fashion.

### Example 2

A fluid composition in the shape of an emulsion obtained from the concentrate of a mixture combining the product of thermal-alkaline hydrolysis of corn (nejayote) and an extract form the alkaline treatment of rice husk, containing the following composition: 15.3 g/L ferulic acid, 0.9 g/L coumaric acid, 0.68 g/L vanillic acid, 180 mg/L protocathecoic acid, as active ingredients for phytoceutical properties, as well as dextrins and starches in a concentration of 114 g/L, Vitamin A 2.1 μg/100 g, Vitamin C 83 mg/100 g, proteins 23 g/L. Said composition was administered into a group of 3 Mastiff, Dalmatian and Great Dane breed dogs with testicular cancer conditions, with positive results (stopping tumor growth). The quality of life of these pets also improved in a remarkable fashion.

### Example 3

A solid composition obtained from dehydrating a concentrate of the mixture combining the product of thermal-alkaline hydrolysis of corn (nejayote) and an extract form the alkaline treatment of rice husk, with following composition:

15% ferulic acid, 1% coumaric acid, 0.6% vanillic acid, 0.25% protocathecoic acid, as active ingredients; furthermore, said composition contains other nutritional elements such as dextrins and starches 60%, Vitamin A 0.35 μg/100 g, Vitamin C <14.00 mg/100 g, proteins 20%, ashes 3%. Said composition was administered to 2 female dogs and one male dog from a litter of nine Saint Bernard breed dogs which were 9 years of age and had a genetic history of breast and cervical cancer in females and testicular cancer in males. The development of cervical cancer and breast cancer in the study females and the development of testicular cancer in the study male were delayed, whereas the other members of the same litter started to develop said cancers at 8 years of age. The quality of life and the appearance of these pets improved remarkably according to the owners of the pets.

### Example 4

The tests of anti-oxidizing ability of the compositions in examples 1, 2 and 3. Said anti-oxidizing ability is the ability which conveys, to a considerable extent, the phytoceutical properties of our composition. By using the supplemented extract of nejayote with 200 ppm of ferulic acid, as shown in the attached chart “Accelerated Oxidization Tests” in (Meq/Kg), it was noted that this formulation had a delaying activity in peroxides higher than the pure ferulic acid and almost similar to a powerful anti-oxidizer such as Butylated Hydroxy Anisole (BHA), thus showing a potential for cancer prevention when this occurs due to the formation of reactive types of oxygen.

| | | | | Supplemented | |
|---|---|---|---|---|---|
| | | | | Extract | |
| | | Pure | Pure | (Nejayote | |
| | Control | Ferulic | Coumaric | 1000 ppm + | |
| Time | (pure BHA) | Acid | Acid | 200 ppm Pure | Control |
| (Hs) | 200 ppm | (200 ppm) | (200 ppm) | Ferulic Acid) | (Pure Oil) |
| 0 | 0.00 | 0.00 | 0.00 | 0.00 | 0.00 |
| 4 | 0.00 | 0.00 | 0.00 | 0.00 | 0.00 |
| 8 | 0.00 | 0.00 | 0.00 | 0.00 | 1.12 |
| 16 | 0.00 | 0.00 | 0.00 | 0.00 | 9.19 |
| 24 | 0.00 | 0.00 | 5.16 | 0.00 | 31.17 |
| 32 | 0.00 | 3.17 | 26.18 | 0.00 | 52.17 |
| 40 | 0.00 | 4.19 | 46.32 | 1.18 | 52.42 |
| 48 | 0.60 | 7.25 | 31.17 | 6.17 | 17.11 |
| 56 | 0.68 | 13.61 | 1.17 | 8.16 | 0.00 |
| 64 | 1.09 | 20.18 | 0.00 | 9.61 | 0.00 |
| 72 | 0.91 | 21.12 | 0.00 | 15.14 | 0.00 |
| 80 | 1.89 | 24.19 | 0.00 | 16.16 | 0.00 |
| 88 | 8.03 | 31.16 | 0.00 | 30.17 | 0.00 |
| 96 | 41.94 | 36.19 | 0.00 | 41.06 | 0.00 |
| 104 | 45.11 | 51.14 | 0.00 | 54.01 | 0.00 |
| 112 | 36.12 | 31.05 | 0.00 | 36.12 | 0.00 |
| 120 | 24.10 | 21.05 | 0.00 | 7.19 | 0.00 |
| 128 | 6.16 | 0.00 | 0.00 | 0.36 | 0.00 |

The determination was performed by AOAC Official Method 965.33/(AOCS Method Cd 8-53) 2000 AOAC International.

What is claimed is:

1. A phytoceutical composition useful for the treatment and/or prevention for degenerative diseases selected of:
a) breast cancer and cervical cancer in females;
b) testicular cancer in males; and
c) skin cancer in both females and males;
in pet dogs, comprising active ingredients having cinnamic acid derivatives, wherein said cinnamic acid derivatives are obtainable from the cooking liquor (nejayote) from corn grains in the nixtamalization process, strengthened with concentrates of ferulic acid crystals obtained from said cooking liquor (nejayote).

2. The phytoceutical composition according to claim 1, wherein one or more of said active ingredients are selected from the group of phenolic compounds consisting of:
trans-ferulic acid,
p-coumaric acid,
protocathecoic acid,
vanillic acid,
caffeic acid,
chlorogenic acid, and
sinapinic acid,

3. The phytoceutical composition according to claim 2, wherein said active ingredients contain at least two active phenolic compounds having a synergic or additive activity.

4. The phytoceutical composition according to claim 1, wherein said composition further comprises one or more solids selected from the group consisting of:
fructose, sucrose, dextrins, glucose, starches, proteins, soluble and insoluble fiber, and inert inorganic compounds.

5. The phytoceutical composition according to claim 4, wherein said composition is in form of a solid or a liquid.

6. The phytoceutical composition according to claim 1, wherein said composition further comprises one or more alkaline extracts from different plant species containing derivates of cinnamic acid.

7. The phytoceutical composition according to claim 6, wherein said alkaline extracts are alkaline extracts from plant species selected from the group consisting of: corn, rice, cane bagasse, oat, wheat, barley, sugar beet, tomato, citric fruits, vegetables, grass, stubble, and coffee.

8. The phytoceutical composition according to claim 7, wherein said alkaline extract is alkaline extract from rice.
